(19) United States
(12) Patent Application Publication
(10) Pub. No.: US 2004/0147932 A1
(43) Pub. Date: Jul. 29, 2004

(54) Device for performing automated microfracture

(76) Inventors: Brian Burkinshaw, Pflugerville, TX (US); Lance Terrill, Gainesville, FL (US)

Correspondence Address: ZIMMER TECHNOLOGY, INC., 150 N. WACKER DRIVE, SUITE 1200, CHICAGO, IL, 60606, US

(21) Appl. No.: 10/697,068

(22) Filed: Oct. 30, 2003

Related U.S. Application Data
(63) Continuation-in-part of application No. 10/348,507, filed on Jan. 21, 2003.
(60) Provisional application No. 60/418,545, filed on Oct. 15, 2002.

(51) Int. Cl. A61B 17/16

(52) U.S. Cl. 606/79

(57) ABSTRACT

The present invention is directed to a method for repair defects in articular cartilage, and, more particularly, to a new method for performing automated microfracture surgery on subchondral bone to repair articular cartilage. The microfractured holes on the surface of the subchondral bone are formed with an automated process using a pneumatically driven orthopedic microfracture instrument. The instrument moves a fracture pin through the end of a guide tube until a sharp end of the fracture pin punctures or penetrates the subchondral bone plate and creates a microfracture or hole in the bone.

### CROSS-REFERENCED TO RELATED APPLICATION

This application is a continuation-in-part of co-pending application Ser. No. 10/348,507, filed Jan. 21, 2003.

### BACKGROUND OF THE INVENTION

1. Field of the Invention

The present invention is generally directed to a device for repairing defects in articular cartilage, and, more particularly, to a device for performing automated microfracture on subchondral bone to repair articular cartilage.

2. Description of the Related Art

Articular cartilage is a highly organized avascular tissue composed of chondrocytes formed in an extracellular matrix. This tissue is extremely important to the normal, healthy function and articulation of joints. Articular cartilage enables joint motion surfaces to articulate smoothly with a very low coefficient of friction. It also acts as a cushion to absorb compressive, tinsile, and shearing forces and, thus, helps protect the ends of bone and surrounding tissue.

Injuries and defects to articular cartilage are frequent. Traumatic chondral injuries, for example, are common in sports and other activities that cause severe stress and strain to joints. Osteoarthritis is also a common condition that develops as cartilage wears, weakens, and deteriorates at the joint motion surfaces of bones.

Unfortunately, articular cartilage is generally thin with an extremely low or insignificant blood flow and, as such, has a very limited ability to repair or heal itself. Partial-thickness chondral defects, for example, cannot spontaneously heal. If these defects are left untreated, they often degenerate at the articular surface and progress to osteoarthritis. Full-thickness defects that penetrate subchondral bone can undergo some spontaneous repair if fibrocartilage forms at the defect. Even in spite of the formation of fibrocartilage, clinical evidence shows that full-thickness defects continue to degenerate and progress to osteoarthritis if these defects are left untreated.

Early diagnosis and treatment are crucial to hindering or stopping the progression of arthritis and degeneration of articular cartilage at joint motion surfaces. Today, depending on the grade of chondral damage, patients usually have several surgical options to repair or regenerate articular cartilage.

For small injuries, such as partial-thickness defects, a patient can be treated with a palliative procedure using known lavage and debridement techniques. These techniques remove loose debris and smooth shredded or frayed articular cartilage. Although this arthroscopic technique is common, relief for the patient can be incomplete and temporary.

Osteochondral autologous transplantation (OATS) and autologous chondrocyte implantation (ACI) are two other treatment modalities used to treat larger or more severe articular defects.

In OATS, cartilage is removed from a normal, healthy location and transferred or planted to the defective area. This procedure is inherently limited to the amount or availability of healthy autologous osteochondral grafts in the patient. Spaces between graft plugs and lack of integration with donor and recipient hyaline cartilage are other clinical concerns with OATS.

In ACI, articular cartilage cells are arthroscopically removed or harvested from the patient and sent to a laboratory. Here, the cells are cultured and multiplied. The newly grown chondrocytes are then re-implanted back into the patient at the defective area. The process of growing cells outside the patient can be expensive. Further, this procedure can require a relatively large incision to place the cartilage cells. What's more, several years may be required for the implanted cells to mature fully.

Microfracture is another treatment modality used to treat articular defects. This technique is a marrow stimulating arthroscopic procedure to penetrate the subchondral bone to induce fibrin clot formation and the migration of primitive stem cells from the bone marrow into the defective cartilage location. More particularly, the base of the defective area is shaved or scraped to clear away debris and loose tissue. An arthroscopic awl or pick is then used to make small holes or microfractures in the subchondral bone plate to induce bleeding. The end of the awl is manually struck with a mallet to form the holes while care is made not to penetrate too deeply and damage the subchondral plate. The holes penetrate a vascularization zone and stimulate the formation of a fibrin clot containing pluripotential stem cells. The clot fills the defect and matures into fibrocartilage.

Microfracturing the subchondral bone plate can be a successful procedure for producing fibrocartilaginous tissue and repairing defective articular cartilage. The current procedure or method for performing the surgical technique, though, has some disadvantages. As one disadvantage, the microfractures or holes are made when the surgeon manually strikes or otherwise forces the awl into the subchondral bone plate. Specifically, the holes are manually created. Manually created holes in the bone plate can have inconsistent depths depending on the force applied to the awl. If the holes are not deep enough, then the formation of the fibrin clot may not occur. On the other hand, if the holes are too deep, then the subchondral bone plate can be damaged and lead to unwanted consequences and complications. The depth of the holes, thus, depends on the skill of the surgeon to accurately and consistently hit the end of the awl and force it to the correct depth in the bone plate.

As another disadvantage, many microfractures may be placed in a single surgery. Each hole must be manually placed and accurately spaced apart. The creation of the many holes can take significant time during the surgery. Depending on the size of the defect being treated, 25-100 or more holes could be required. An hour or more may be required to manually place these holes.

As yet another disadvantage, the microfractures should be placed 3-4 mm apart from each other on the bone plate. The placement of these holes and distance between adjacent holes, thus, depends on the visual judgment and skill of the surgeon.

It therefore would be advantageous to provide a new method and device for performing the microfracture surgical technique. Such a method and device would eliminate the disadvantages associated with conventional microfracture surgery.

### SUMMARY OF THE INVENTION

The present invention is generally directed to a device for performing automated microfracture, and methods of using such a device. In one illustrative embodiment, the device for forming multiple holes in subchondral bone comprises a housing, a fracture pin having a sharpened tip, the sharpened tip adapted to penetrate subchondral bone, and a trigger that is adapted to, when actuated, cause the sharpened tip to move and penetrate into the subchondral bone, thereby forming at least one of the holes.

In another illustrative embodiment, the device for forming multiple holes in subchondral bone comprises a housing, a fracture pin having a sharpened tip, the sharpened tip adapted to penetrate subchondral bone, and an acuatable cylinder that is adapted to, when actuated, cause the sharpened tip to move and penetrate into the subchondral bone, thereby forming at least one of the holes.

In yet another illustrative embodiment, the device for forming multiple holes in subchondral bone comprises a housing, a fracture pin having a sharpened tip, the sharpened tip adapted to penetrate subchondral bone, and a movable hammer that is adapted to, when actuated, cause the sharpened tip to move and penetrate into the subchondral bone, thereby forming at least one of the holes.

In a further illustrative embodiment, the device for forming multiple holes in subchondral bone comprises a housing, a fracture pin having a sharpened tip, the sharpened tip adapted to penetrate subchondral bone, and means for causing the sharpened tip to move and penetrate into the subchondral bone, thereby forming at least one of the holes.

### BRIEF DESCRIPTION OF THE DRAWINGS

The invention may be understood by reference to the following description taken in conjunction with the accompanying drawings, in which like reference numerals identify like elements, and in which:

FIG. 1 is a perspective view of a pneumatically powered microfracture inserter that has a microfracture pin assembly of the present invention;

FIG. 2 is an exploded view of the microfracture pin assembly of FIG. 1;

FIG. 3 is an assembled view of the microfracture pin assembly of FIG. 2 with a delivery angle on the guide tube which may have a range between 30-45 degrees;

FIG. 4 is an alternate embodiment for a guide tube and fracture pin of the microfracture pin assembly with a delivery angle on the guide tube which may have a range between 30-45 degrees;

FIG. 5 is another alternate embodiment for a guide tube and fracture pin of the microfracture pin assembly with a delivery angle on the guide tube which may have a range between 45-60 degrees;

FIGS. 6-8 depict one illustrative embodiment of an automated microfracture tool in accordance with the present invention; and

FIGS. 9-11 depict yet another illustrative embodiment of an automated microfracture tool in accordance with the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the invention are described below. In the interest of clarity, not all features of an actual implementation are described in this specification. It will, of course, be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present invention will now be described with reference to the attached drawings. which are included to describe and explain illustrative examples of the present invention. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

The instruments, method and steps of the present invention are now described in more detail. The method describes the steps to perform an automated surgical microfracture procedure on subchondral bone to repair or regenerate articular cartilage at a full-thickness defect. Some of these steps described in the method are known to those skilled in the art and will not be discussed in great detail. Further, one skilled in the art will appreciate that certain steps may be altered or omitted while other steps may be added without departing from the scope of the invention.

Further, the novel microfracture method of the present invention will be described in connection with arthroscopic knee surgery, though one skilled in the art will appreciate that the microfracture method may be done as an “open” procedure as well. Specifically, the method will address a patient having unstable cartilage covering the underlying bone or a full-thickness defect (i.e., loss of articular cartilage down to the bone), for example, in either a weight bearing area of contact between the femur and tibia or in an area of contact between the articular surface of the patella and the trochlear groove. One skill in the art, though, will appreciate that the invention can be utilized at various other locations other than the knee to repair or regenerate articular and/or fibro cartilage.

To facilitate a discussion of the present invention, the method is divided into three different sections: Diagnostic Evaluation; Site Preparation; and Microfracture Technique and Instrumentation. Each of these sections is discussed seriatim.

Diagnostic Evaluation

Once on the operating table, the patient is placed in a supine position, and standard arthroscopic portals are made through the skin. Generally, two or more ports are made to provide access to the knee. An arthroscopic camera is used through one port, and other arthroscopic instrumentation are used through the other port or ports.

Any associated pathology, such as meniscal tears or loose body, should be addressed before the microfracture procedure. If no such conditions exist, a thorough diagnostic examination of the knee is performed. This examination should include an inspection of the suprapatellar pouch, the medial and lateral gutters, the patellofemoral joint, and the notch and its contents. Further, the examination can include the medial and lateral compartments and posterior horns of both menisci. Other intra-articular procedures and examinations, as deemed necessary, can also be included. A thorough examination may be helpful when a loss in visualization occurs after fat droplets and blood enter the knee from the microfractures.

Site Preparation

The next step is to identify visually the lesion or defect in the articular cartilage. The boundaries or limits of the defect should be clearly defined. Next, the exposed bone under the defect is debrided of cartilage tags. A curette and full radius resecter or “Gator” shaver can be used for debridement. All loose or marginally attached cartilage from the surrounding rim of articular cartilage should be debrided to create a stable edge of healthy, viable cartilage around the defect.

The creation of an edge has an important purpose: It provides a pool or recess to receive the formation of a clot. Further, a curette may be used to remove the calcified layer of cartilage from the base of the defect. Removal of this calcified layer is important as it enhances the amount of defect that is ultimately filled. Removal of this layer also provides a more adequate surface for adherence of the clot and for improved chondral nutrition through subchondral diffusion. Additionally, care should be taken not to debride through the calcified layer to avoid excessive damage to the subchondral bone.

Microfracture Technique and Instrumentation

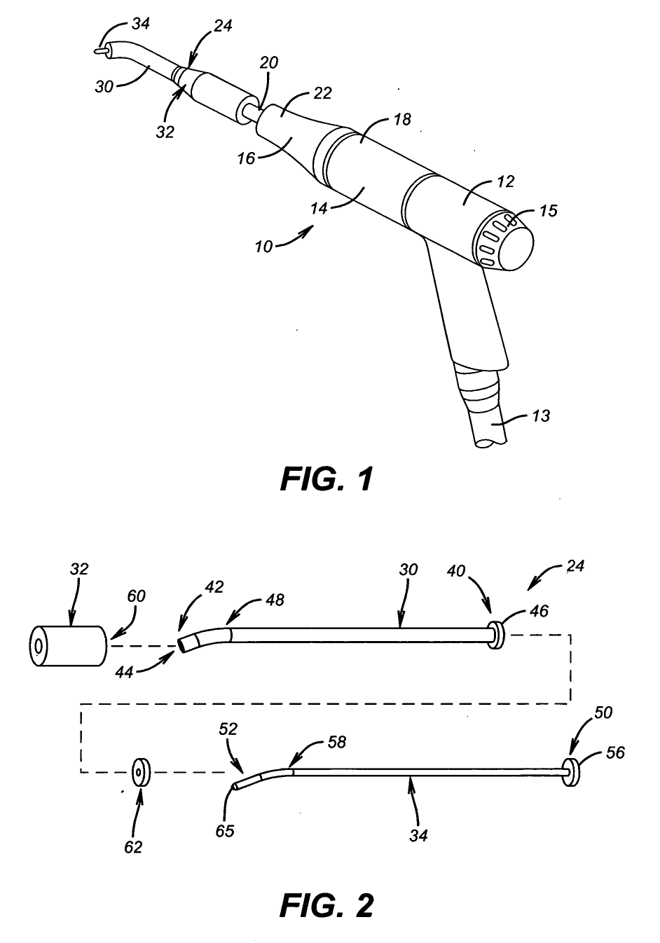

The important advantage of the present invention is that the microfractured holes on the surface of the subchondral bone plate are formed with an automated process. FIG. 1 shows a pneumatically driven orthopedic microfracture instrument or inserter 10. The microfracture inserter 10 generally comprises a handle 12 to which an air hose 13 is attached. The handle 12 supports or connects to a cylinder impaction assembly 14 at one end and a flow control knob 15 at another end. The control knob 15 is adapted to control and adjust the flow of air into the handle 12 and the impaction assembly 14. A nose assembly 16 connects to a distal end 18 of the impaction assembly 14. A shaft 20 protrudes from a distal end 22 of the nose assembly 16 and removably connects to a microfracture pin assembly 24. This assembly 24 generally includes a guide tube 30, a connector 32 and a fracture pin 34.

The present invention centers around the microfracture pin assembly 24 and its use in microfracture surgical techniques. This assembly 24 can be attached to or used in conjunction with various types of automated orthopedic guns or instruments known in the art, such as pneumatic, hydraulic, or electric powered orthopedic guns and instruments. A pneumatically driven orthopedic gun (such as orthopedic microfracture inserter 10) is just one example. Generally described though, pressured air is pumped via the air hose 13 and into a manifold in the housing 12. The manifold abuts an anvil or anvil plate. The manifold, anvil, valves and other components function to move a cylinder or piston in the cylinder impaction assembly 14. The piston is driven with air pressure to impact against one end of the fracture pin 34. As the piston strikes the end, the fracture pin 34 is guided along the inside of the guide tube 30 until a sharp end of the fracture pin 34 moves outwardly, away from the end of the guide tube 30. This sharp end punctures or penetrates the subchondral bone plate and creates a microfracture or hole in the bone.

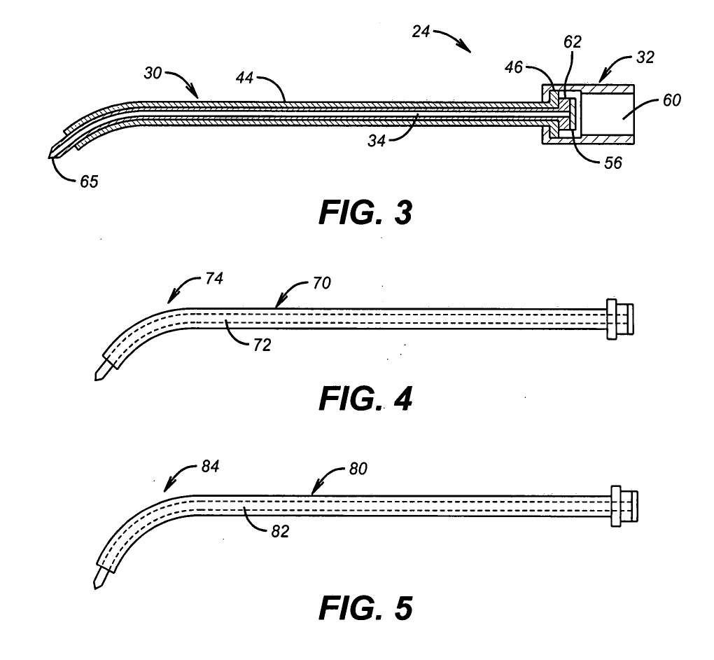

Turning now to FIGS. 2 and 3, the microfracture pin assembly 24 is shown in more detail. As noted, the pin assembly 24 includes a guide tube 30, a connector 32 and a fracture pin 34. The guide tube 30 has an elongated cylindrical configuration with a body that extends from a proximal end 40 to a distal end 42. A cylindrical bore 44 extends completely through the body from the proximal to distal ends. The proximal end 40 includes a head portion 46 and the distal end 42 includes an angled tip 48.

The flexible fracture pin 34 has an elongated cylindrical shape with a body that extends from a proximal end 50 to a distal end 52. The proximal end 50 includes a head portion 56, and the distal end 52 included an angled tip 58. The flexible fracture pin 34 is sized and shaped to slideably fit into and move in bore 44 of the guide tube 30.

Once assembled, the proximal ends 40, 50 of the guide tube and fracture pin 34 are positioned in a cavity or recess 60 of the connector 32. A spring or biasing member 62 is placed between the head 46 of the guide tube 30 and the head 56 of the fracture pin 34. The biasing member 62 provides the restriction force needed to withdraw the fracture pin 34 from the bone and return the pin to the “ready” position in preparation for the next automated strike.

In operation, the piston (or other mechanism) of an automated orthopedic gun or instrument strikes the head 56 of the fracture pin 34. The fracture pin 34 forceably moves down through the bore 44. As the pin moves, the spring 62 compresses until the head 56 of the fracture pin 34 and the head 46 of the guide tube 30 are brought into close proximity of each other. Simultaneously, a sharp tip 65 of the fracture pin 34 extends outwardly from the end of the guide tube 30.

As noted, the distal end 42 of the guide tube 30 includes an angled tip 48. The angulation of this tip may vary. FIG. 3, for example, shows a 30 degree angle. FIG. 4 shows an alternate embodiment for the guide tube 70 and fracture pin 72 wherein the guide tube 70 has an angled tip 74 with an angle of approximately 45 degrees. FIG. 5 shows another alternate embodiment for the guide tube 80 and fracture pin 82 wherein the guide tube 80 has an angled tip 84 with an angle of approximately 60 degrees. Other angles are also within the scope of the present invention. Guide tubes, for example, can be provided to have angled tips of various degrees between about 1 and 90 degrees.

The microfracture pin assembly and the automated orthopedic gun or instrument can be designed to be disposable or re-useable. Further, one skilled in the art will appreciate that various materials can be used to fabricate the pin assembly and the automated orthopedic gun or instrument. The guide tube, for instance, can be made of polymer while the fracture pin is made of spring steel, Nitinol® or other acceptable and durable material and designed to be flexible and fracture resistant for the designed application and duration of use. Further yet, the spring can be formed as a coiled compression spring, a wave spring, rubber-like bumper, or other biasing member known in the art.

As another advantage of the present invention, the connector 32 is adapted to be attached and detached from the microfracture insert 10 (FIG. 1). As such, the guide tube, biasing member, and fracture pin can be easily changed during a microfracture procedure. Various guide tubes with different angled tips or different fracture pins can be used in the same procedure. The connector can be designed with a bayonet or similar type of quick-connect feature to aid the surgeon or assistant during changes of the tip and/or guide tubes during the procedure.

During the microfracture surgical procedure, the microfracture instruments and microfracture pin assembly are used to create multiple holes or microfractures in the exposed subchondral bone plate. These holes can be formed in close proximity to each other. Preferably, though, adjacent holes should not break into each other since the subchondral bone plate should not be damaged. Microfractured holes, for example, can be placed approximately 3-4 mm apart.

The depth of the holes can very slight, from about 2-4 mm. Generally, an adequate depth is reached when the subchondral bone plate is penetrated just enough to release fat droplets.

Another advantage of the present invention is that the microfracture pin assembly includes a stop mechanism designed to regulate and limit the depth at which the bone plate is penetrated. Looking to FIGS. 2 and 3, the depth of the microfractures is equal to the travel of the fracture pin 34 inside the guide tube 30. The fracture pin 34, though, is limited in movement or travel since it is designed to move down the guide tube 30 a distance equal to the compression of the spring 62. In other words, as the spring 62 compresses and the heads 56 and 46 move together, the fracture pin 34 moves out from the distal end of the guide tube 30. The fracture pin 34 is prevented from moving too far since the head 46 of the guide tube 30 will abut against the head 56 and stop the fracture pin 34 from moving. These two heads, in combination with spring 62, thus, act as a safety mechanism and limit the amount of travel of the fracture pin 34.

The spring 62 can be sized and shaped and selected to have specific biasing properties so the fracture pin 34 extends about 2-4 mm from the distal end of the guide tube 30 when activated with the microfracture instrument. Different springs can be used to vary the travel of the fracture pin 34 and, thus, vary the depth of the microfractures in the bone plate.

Microfractures should first be placed around the periphery or edge of the defect and immediately adjacent to healthy cartilage rim. The holes can be placed in a peripheral pattern working towards the center of the defect (as described by Steadman and others skilled in this procedure).

The number and spacing of microfractures should be sufficient to establish a super clot. Such a clot will provide an optimal environment for a viable population of pluripotential marrow cells (messenchymal stem cells) to differentiate into stable tissue within the lesion or defect.

Another advantage of the present invention is that consistent and accurate spacing between adjacent microfractures can be obtained. The distal end 42 of the guide tube 30 can function as a guide for the placement of holes in the bone. In particular, the distal end 42 of the guide tube 30 has a diameter between about 6-8 mm. During the surgical procedure, after a first hole is made in the subchondral bone plate, the guide tube 30 is moved until the outer perimeter of the distal end 42 is adjacent the perimeter of the first hole. A second hole can now be made with the edge of the guide tube 30 adjacent the first hole. This second hole will be spaced about 3-4 mm (i.e., about one-half of the diameter of the guide tube 30) from the first hole. In this manner, the surgeon ensures that successive holes are evenly spaced apart.

Guide tubes can be made to have different diameters to provide different spacing between adjacent holes. The different diameters can have a wide range, depending on the diameter of the fracture pin, the microfracture procedure and preferences of the surgeon.

Another important advantage of the present invention is that the microfractures are not manually made. Instead, the microfractures are formed with an automated process using, for example, the pneumatic instrument discussed in connection with FIG. 1. These microfiactures can be quickly and easily created with a simple activation of the instrument. A surgeon merely pulls a trigger, pushes a button, steps on a control switch, or performs a similar task to activate movement of the fracture pin and create a microfracture in the bone plate. Successive microfractures are created with repeated activation of the instrument.

As yet another advantage of the present invention, the microfracture procedure of the present invention is simpler to perform. During some prior microfracture knee procedures, three arthroscopic portals were made in the skin of the patient. An arthroscopic camera was inserted into one port; a fluid management device was inserted into the second port; and microfracture instruments, such as awls or picks, were inserted through the third port. A primary assistant would hold the camera and pass instruments to the surgeon. The surgeon, in turn, needed two hands to create the microfractures: one hand held the awl, and one hand held the mallet to strike the awl. With the present invention, two hands are not required to create a microfracture. The surgeon can hold the pneumatic instrument with one hand and activate the instrument with the same hand or a foot. As such, the second hand of the surgeon can occupy another task, such as holding and manipulating the camera.

Once all of the microfractures are placed, the arthroscopic fluid pump pressure is reduced. Under direct visualization, the fat droplets and blood from the microfractured holes can be seen.

After the step of microfracturing the surface of subchondral bone is complete and the release of marrow is adequate, all instruments are removed from the knee. At this time, the joint is evacuated of fluid. No drains should be placed intra-articularly. A super clot rich in marrow elements should be allowed to form and stabilize. The microfracture technique produces a rough surface in the lesion to which the clot can easily adhere while simultaneously maintaining the integrity of the subchondral plate for shaping the joint motion surface. At this time, it may also be appropriate for a protective, biologically compatible coating to be placed over the microfracture site. The purpose of such a coating would be to protect the clot site or even provide a culture bed for stimulating the growth of the repair cartilage. The arthroscopic ports are then closed.

This disclosure will not discuss in detail post-operative protocol or rehabilitation as such procedures are known in the art (Steadman) and tailored to meet the specific needs of the patient. Generally though, the rehabilitation should promote an environment for the pluripotential cells from the marrow to differentiate into articular cartilage cells. A healthy development of these cells will lead to the development and proliferation of durable cartilage that fills the original defect or lesion.

It should be emphasized that although the method of the present invention was described with a specific number and sequence of steps, these steps can be altered or omitted while other steps may be added without departing from the scope of the present invention. As such, the specific steps discussed in the preferred embodiment of the present invention illustrate just one example of how to utilize the novel method and steps of the present invention.

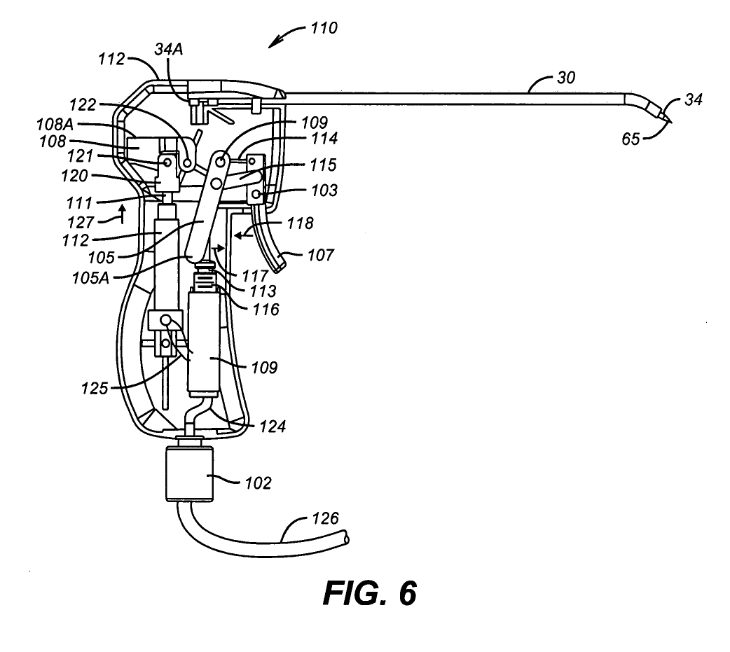

One illustrative embodiment of an automated microfracture instrument in accordance with the present invention is depicted in FIGS. 6-8. As shown therein, the illustrative embodiment of the automated microfracture instrument or inserter 110 is generally comprised of a housing 112 that has a general pistol-shaped configuration. The housing 112 may be made of any of a variety of materials, e.g., plastic, metal, composites, etc. The automated microfracture instrument 110 is further comprised of an air block 109, an air block control lever 113, a spring 116, a structural lever member 105, having an end 105A, a trigger 107, a structural member 119, and a trigger return biasing spring 114. A structural member 115 connects the trigger 107 to the structural lever member 105. An air supply port 102 is operatively coupled to the pneumatic block 109 via a pneumatic supply line 124. Air may be provided to the instrument 110 via an air hose 126 that is coupled to a pressurized air source (not shown). Also depicted in FIGS. 6-8 is a pneumatic cylinder 112 having a cylinder rod 111, a connector 120, and a hammer 108 having a striking surface 108A. A second pneumatic supply line 125 is depicted in FIG. 6.

In the depicted embodiment, the pneumatic cylinder 112 is a dual acting cylinder although a single acting cylinder may be employed in some embodiments of the present invention. A guide tube 30 with a fracture pin 34 positioned therein is also depicted in the drawings. As indicated previously, the fracture pin 34 is operatively coupled to the housing, and the fracture pin 34 is configured and adapted to penetrate the subchondral bone, i.e., the sharpened tip 65 is used to make holes in the subchondral bone. The various components of the microfracture instrument 110 are pinned to one another or to the housing via a variety of pinned connections, e.g., 103, 109, 122, as will be described more fully below. The various components of the automatic microfracture instrument 110 may be made from a variety of materials, e.g., a plastic, metal or composites. Moreover, the various components depicted in FIGS. 6-8 may be sized and configured for the particular application, e.g., the desired configuration of the instrument 110. For example, the stroke of the pneumatic cylinder 112 may vary as may the manner in which the hammer 108 is operatively coupled to the cylinder rod 111. Additionally, the size and configuration of the various mechanical linkages depicted in FIGS. 6-8 may be varied depending upon the spatial relationship between the various components. Nevertheless, the attached drawings provide one illustrative embodiment of an automated microfracture instrument 110 that may be employed in practicing the methods described herein.

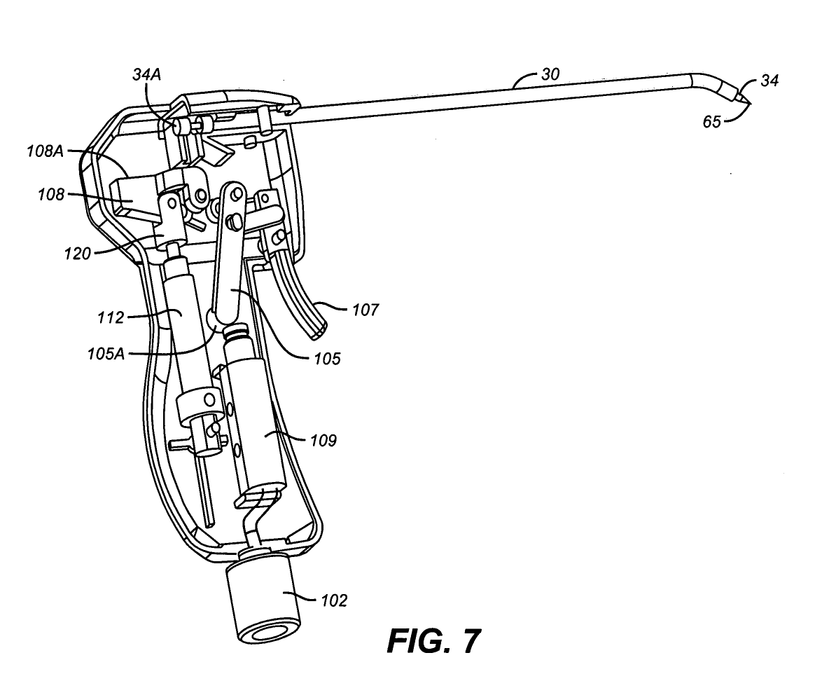

The operation of the illustrative automated microfracture instrument 110 will now be described. The fracture pin 34 is depicted in FIGS. 6 and 7 in its initial, retracted position, i.e., in the position wherein the surgeon would place the sharp tip 65 of the automated microfracture instrument 110 at the desired location within the patient prior to actuating the instrument 110. The trigger 107, when actuated, causes the sharpened tip 65 to move and penetrate the subchondral bone, thereby forming the desired holes. To actuate the instrument 110, the surgeon pulls on the trigger 107, thereby moving the trigger 107 in the direction indicated by the arrow 118. In turn, this causes the upper portion 107A of the trigger mechanism to pivot about the fixed pivot point 103 which, via structural member 115, causes the end 105A of the lever member 105 to move in the direction indicated by the arrow 117. Forward movement of the end 105A urges the control lever 113 of the air block 109 downward, thereby providing a flow path for air from the air hose 126 to flow through the air block 109 and into the pneumatic cylinder 112. Note that the downward movement of the control lever 113 compresses the spring 116, thereby creating a biasing force that will eventually be used to return the control lever 113 to the position depicted in FIG. 6. It should also be noted that the end 105A of the structural lever member 105 and the end 113A of the control lever 113 are essentially cammed surfaces that are configured such that they slidingly engage one another to achieve the movements described herein.

When air is supplied to the pneumatic cylinder 112, the cylinder rod 111 is driven in the direction indicated by the arrow 127. In turn, the hammer 108 pivots about the fixed pivot point 122 due to its pivotal connection, via pivot pin 121, with the connector 120. In turn, the striking surface 108A of the hammer 108 is driven into contact with the striking surface 34A of the fracture pin 34, thereby urging the sharp tip 65 forward to form the desired holes. When the trigger 107 is released, the biasing force created by the spring 114 will return the structural lever member 105 to the position depicted in FIG. 6. Additionally, the biasing force created by the spring 116 will return the control lever 113 to the position depicted in FIG. 6. In this position (i.e., the normally on position), air is supplied to the dual-acting cylinder 112 in such a manner that the cylinder rod 111 and the hammer 108 are returned to the position indicated in FIG. 6.

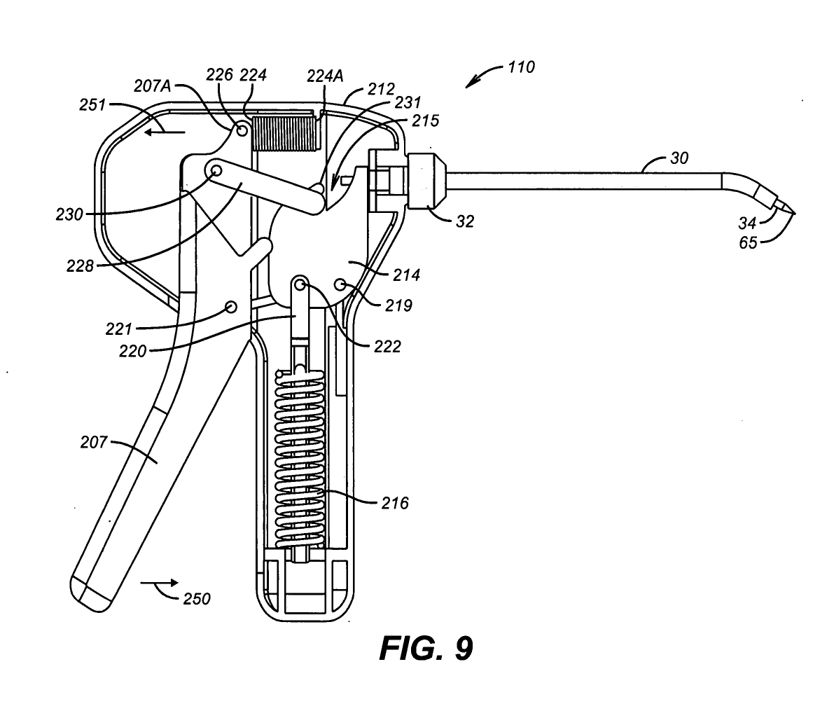

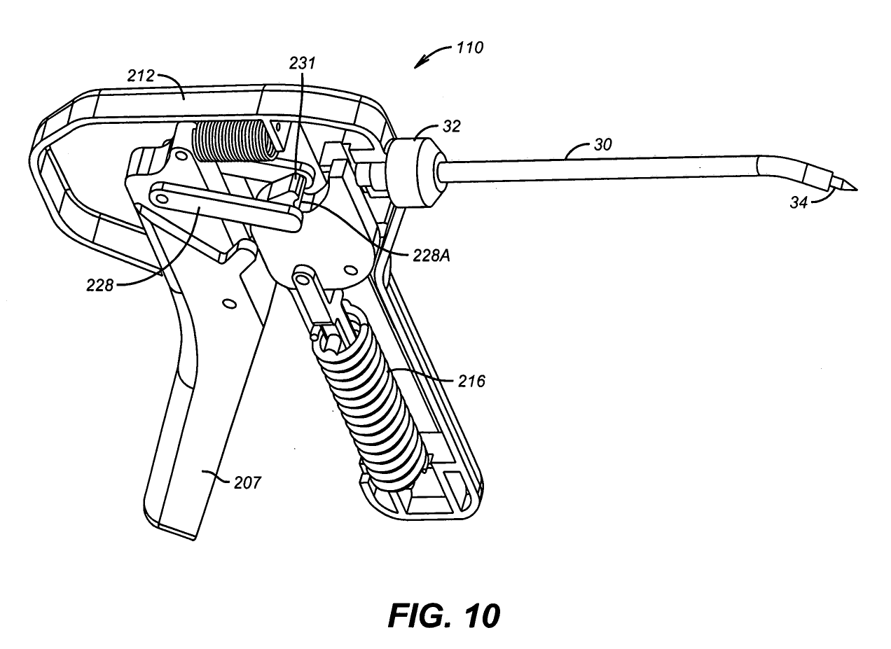

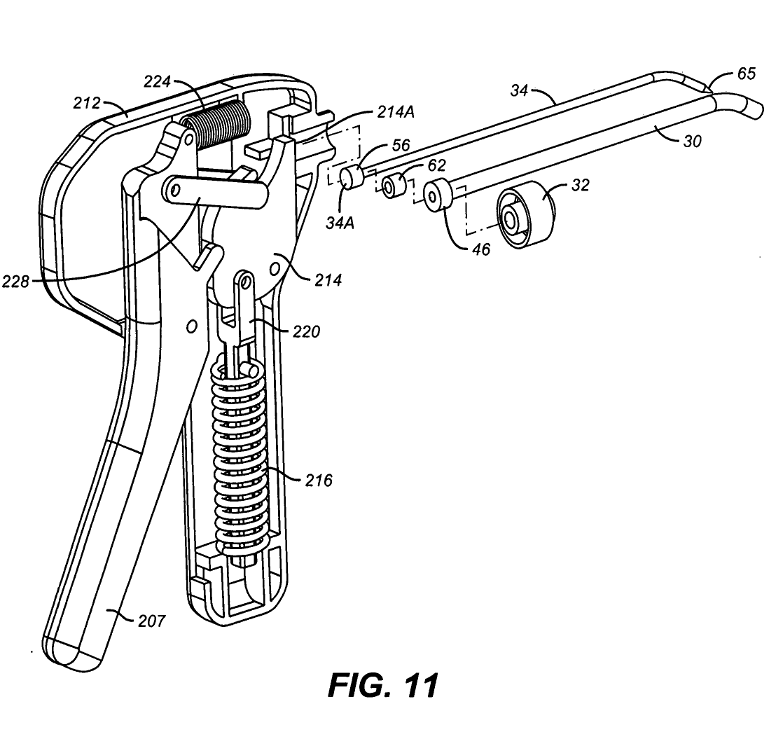

FIGS. 9-11 depict yet another illustrative embodiments of an automated microfracture instrument 10 in accordance with the present invention. As shown therein, the instrument 110 is comprised of a housing 212, a hammer 214, a hammer-biasing spring 216, a trigger 207 and a trigger return spring 224. Also depicted in FIG. 9 is a guide tube 30 and a fracture pin 34, which has a sharpened tip 65. The fracture pin 34 is also operatively coupled to the instrument such that the sharpened tip 65 of the fracture pin 34 may be moved so as to form holes in the subchondral bone. The hammer-biasing spring 216 is operatively coupled to the hammer 214 by arm 220 at pivot pin 222. The hammer 214 is adapted to pivot about the fixed pivot point 219. The trigger 207 is operatively coupled to the trigger return spring 224 at pivot point 226. The end 224A of the trigger return spring 224 is fixedly coupled to the housing 212. An arm 228 provides connection between the trigger 207 and the hammer 214. More specifically, as shown in FIG. 10, the arm 228 has a cross bar 228A that is adapted to be positioned in a recess 215 defined, at least in part, by the body of the hammer 214 and the curved tip or hook 231 of the hammer 214. As with the previous embodiment, the various components of the automated microfracture instrument 110 may be made from a variety of materials, and the physical size and configuration of such components may vary depending upon the particular application and the intended working interrelationship between the various components.

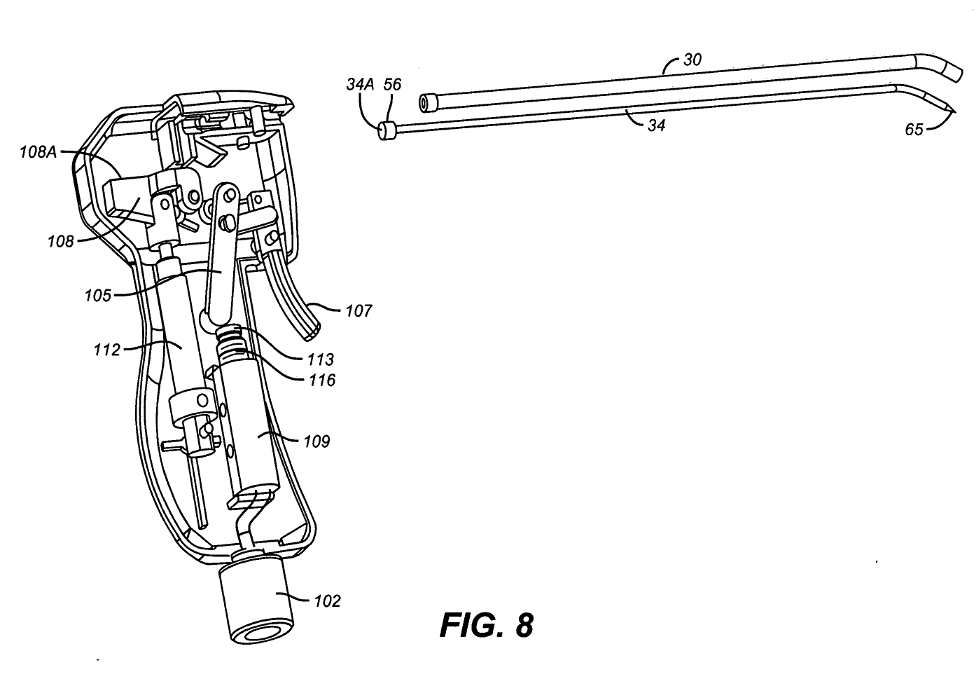

As with the previous embodiment, actuation of the trigger 207 causes the sharpened tip 65 of the fracture pin 34 to move and penetrate the subchondral bone, thereby forming the desired holes. More specifically, when the trigger 207 is moved in the direction indicated by arrow 250, the trigger 207 pivots about the fixed pivot point 221. In turn, the end 207A of the trigger 207 is moved in the direction indicated by the arrow 251. This movement creates a biasing force in the trigger return spring 224 that will eventually cause the end 207A of the trigger 207 to return to the position indicated in FIG. 8. When the trigger 207 is actuated (moved forward in the direction 250), the hammer 214 rotates counter-clockwise around fixed pivot point 219 due to the cross arm 228A being positioned in the recess 215 formed in the hammer 214. The counter-clockwise rotation of the hammer 214 urges the arm 220 downward, thereby creating a biasing force in the spring 216 that will tend to return the hammer 214 to the position depicted in FIG. 9. As the hammer 214 is rotated counterclockwise, the striking surface 214A of the hammer 214 moves away from contact with the striking surface 34A of the fracture pin 34. Continued movement of the trigger 207 in the direction indicated by the arrow 250 causes continued counter-clockwise rotation of the hammer 214 until such time as the cross bar 228A of the arm 228 slips over the hook 231 and out of the recess 215 in the hammer 214, thereby freeing the hammer 214 to rapidly rotate in a clockwise direction due to the return biasing force created by the spring 216. When the hammer 214 is released, the striking face 214A of the hammer 214 engages the striking face 34A of the fracture pin 34 with sufficient force to drive the sharp tip 65 of the fracture pin 34 into the bone, thereby creating the desired holes. Thereafter, the trigger 207 may be released, wherein the return biasing force created in the trigger return spring 224 will cause the end 207A of the trigger 207 to return to the position depicted in FIG. 9. As part of that return movement, the cross bar 228A of the arm 228 will move up and over the hook 231 on the hammer 214 and reposition the cross bar 228A in the recess 215 in the hammer 214.

The present invention is generally directed to a device for performing automated microfracture, and methods of using such a device. In one illustrative embodiment, the device for forming multiple holes in subchondral bone comprises a housing, a fracture pin having a sharpened tip, the sharpened tip adapted to penetrate subchondral bone, and a trigger that is adapted to, when actuated, cause the sharpened tip to move and penetrate into the subchondral bone, thereby forming at least one of the holes.

In another illustrative embodiment, the device for forming multiple holes in subchondral bone comprises a housing, a fracture pin having a sharpened tip, the sharpened tip adapted to penetrate subchondral bone, and an acuatable cylinder that is adapted to, when actuated, cause the sharpened tip to move and penetrate into the subchondral bone, thereby forming at least one of the holes.

In yet another illustrative embodiment, the device for forming multiple holes in subchondral bone comprises a housing, a fracture pin having a sharpened tip, the sharpened tip adapted to penetrate subchondral bone, and a movable hammer that is adapted to, when actuated, cause the sharpened tip to move and penetrate into the subchondral bone, thereby forming at least one of the holes.

In a further illustrative embodiment, the device for forming multiple holes in subchondral bone comprises a housing, a fracture pin having a sharpened tip, the sharpened tip adapted to penetrate subchondral bone, and means for causing the sharpened tip to move and penetrate into the subchondral bone, thereby forming at least one of the holes.

In one illustrative embodiment, the means for causing the sharpened tip to move and penetrate into the subchondral bone further comprises a hammer that is pivotally coupled to a rod of the cylinder, an air block that is operatively coupled to the cylinder, and a trigger that is operatively coupled to the air block, wherein, when the trigger is actuated, the air block allows air to flow to the cylinder through the air block to thereby actuate the cylinder.

In another illustrative embodiment, the means for causing the sharpened tip to move and penetrate into the subchondral bone comprises a movable hammer that is adapted to, when actuated, cause the sharpened tip to move and penetrate into the subchondral bone, thereby forming at least one of the holes.

In yet another illustrative embodiment, the means for causing the sharpened tip to move and penetrate into the subchondral bone further comprises a cylinder coupled to the moveable hammer.

In a further illustrative embodiment, the means for causing the sharpened tip to move and penetrate into the subchondral bone further comprises a trigger that is operatively coupled to the hammer, the hammer being rotatably moveable by actuation of the trigger, and a hammer biasing spring being operatively coupled to the hammer.

The particular embodiments disclosed above are illustrative only, as the invention may be modified and practiced in different but equivalent manners apparent to those skilled in the art having the benefit of the teachings herein. For example, the process steps set forth above may be performed in a different order. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. A device for forming multiple holes in subchondral bone, comprising:
a housing;
a fracture pin having a sharpened tip, said sharpened tip adapted to penetrate subchondral bone; and
a trigger that is adapted to, when actuated, cause said sharpened tip to move and penetrate into said subchondral bone, thereby forming at least one of said holes.

2. The device of claim 1, wherein said fracture pin is operatively coupled to said housing.

3. The device of claim 1, wherein said device further comprises a biasing member that is adapted to cause said sharpened tip to return to an initial, retracted position after said at least one hole has been formed.

4. The device of claim 1, further comprising a cylinder that is actuated by actuating said trigger.

5. The device of claim 4, further comprising:
a hammer that is pivotally coupled to a rod of said cylinder, said hammer having a striking face; and
a striking face on an end of said fracture pin opposite said sharpened tip, wherein, when said cylinder is actuated, said striking face of said hammer strikes said striking face of said fracture pin, thereby causing said sharpened tip to move.

6. The device of claim 4, wherein said cylinder is a pneumatic cylinder.

7. The device of claim 6, further comprising an air block that is operatively coupled to said pneumatic cylinder, wherein, when said trigger is actuated, said air block allows air to flow to said pneumatic cylinder through said air block, thereby actuating said pneumatic cylinder.

8. The device of claim 7, wherein said trigger is operatively coupled to said air block by a structural bar, one end of said bar being adapted to engage a control lever on said air block at least when said trigger is actuated.

9. The device of claim 1, further comprising a trigger biasing spring coupled to said trigger and said housing, said trigger biasing spring adapted to, when said trigger is actuated, create a biasing force to return said trigger to an initial starting position.

10. The device of claim 4, wherein said cylinder is positioned within said housing.

11. The device of claim 6, wherein said pneumatic cylinder and an air block that is operatively coupled to said pneumatic cylinder are both positioned within said housing.

12. The device of claim 1, further comprising a guide tube having an angled tip, at least a portion of said fracture pin being positioned within said guide tube.

13. The device of claim 1, wherein said fracture pin is removably coupled to said housing and said fracture pin is disposable.

14. The device of claim 1, further comprising means for limiting the movement of said sharpened tip when said trigger is actuated to thereby limit a depth of said at least one hole.

15. A device for forming multiple holes in subchondral bone, comprising:
a housing;
a fracture pin having a sharpened tip, said sharpened tip adapted to penetrate subchondral bone; and
an acuatable cylinder that is adapted to, when actuated, cause said sharpened tip to move and penetrate into said subchondral bone, thereby forming at least one of said holes.

16. The device of claim 15, wherein said fracture pin is operatively coupled to said housing.

17. The device of claim 15, wherein said device further comprises a biasing member that is adapted to cause said sharpened tip to return to an initial, retracted position after said at least one hole has been formed.

18. The device of claim 15, further comprising:
a hammer that is pivotally coupled to a rod of said cylinder, said hammer having a striking face; and
a striking face on an end of said fracture pin opposite said sharpened tip, wherein, when said cylinder is actuated, said striking face of said hammer strikes said striking face of said fracture pin, thereby causing said sharpened tip to move.

19. The device of claim 15, wherein said actuatable cylinder is a pneumatic cylinder.

20. The device of claim 19, further comprising:
an air block that is operatively coupled to said pneumatic cylinder; and
a trigger that is operatively coupled to said air block, wherein, when said trigger is actuated, said air block allows air to flow to said pneumatic cylinder through said air block, thereby actuating said pneumatic cylinder.

21. The device of claim 20, wherein said trigger is operatively coupled to said air block by a structural bar, one end of said bar being adapted to engage a control lever on said air block at least when said trigger is actuated.

22. The device of claim 20, further comprising a trigger biasing spring coupled to said trigger and said housing, said trigger biasing spring adapted to, when said trigger is actuated, create a biasing force to return said trigger to an initial starting position.

23. The device of claim 18, wherein said hammer is pivotally coupled to said housing.

24. The device of claim 20, wherein said trigger is pivotally coupled to said housing.

25. The device of claim 15, wherein said cylinder is positioned within said housing.

26. The device of claim 19, wherein said pneumatic cylinder and an air block that is operatively coupled to said pneumatic cylinder are both positioned within said housing.

27. The device of claim 15, further comprising a guide tube having an angled tip, at least a portion of said fracture pin being positioned within said guide tube.

28. The device of claim 15, wherein said fracture pin is removably coupled to said housing and said fracture pin is disposable.

29. The device of claim 27, wherein said angled tip may have an angle that ranges from approximately 30-60 degrees.

30. The device of claim 27, wherein said guide tube has an outside diameter that ranges from approximately 6-8 millimeters.

31. The device of claim 15, further comprising means for limiting the movement of said sharpened tip when said cylinder is actuated to thereby limit a depth of said at least one hole.

32. A device for forming multiple holes in subchondral bone, comprising:
a housing;
a fracture pin having a sharpened tip, said sharpened tip adapted to penetrate subchondral bone; and
a movable hammer that is adapted to, when actuated, cause said sharpened tip to move and penetrate into said subchondral bone, thereby forming at least one of said holes.

33. The device of claim 32, wherein said hammer is operatively coupled to a cylinder, said hammer being rotationally movable when said cylinder is actuated.

34. The device of claim 32, wherein said hammer is operatively coupled to a trigger, said hammer being rotatably movable when said trigger is actuated.

35. The device of claim 34, wherein said hammer is coupled to a biasing member, said biasing member adapted to create a bias force when said hammer is rotatably moved by actuation of said trigger.

36. The device of claim 34, wherein said device further comprises a trigger biasing member that is operatively coupled to said trigger and said housing, said trigger biasing member being adapted to, when said trigger is actuated, create a biasing force to return said trigger to an initial position.

37. The device of claim 34, further comprising:
a recess formed in said hammer; and
a structural member coupled to said trigger, a portion of said structural member being positioned in said recess in said hammer.

38. The device of claim 34, wherein said structural member comprises a cross bar, and said cross bar is adapted to be positioned in said recess in said hammer.

39. The device of claim 38, wherein said cross bar is sliding removable from said recess after said hammer has been rotated a sufficient distance.

40. The device of claim 32, wherein said fracture pin is operatively coupled to said housing.

41. The device of claim 32, wherein said device further comprises a biasing member that is adapted to cause said sharpened tip to return to an initial, retracted position after said at least one hole has been formed.

42. The device of claim 33, further comprising:
a rod of said cylinder that is pivotally coupled to said hammer, said hammer having a striking face; and
a striking face on an end of said fracture pin opposite said sharpened tip, wherein, when said cylinder is actuated, said striking face of said hammer strikes said striking face of said fracture pin, thereby causing said sharpened tip to move.

43. The device of claim 33, wherein said cylinder is a pneumatic cylinder.

44. The device of claim 43, further comprising:
an air block that is operatively coupled to said pneumatic cylinder; and
a trigger that is operatively coupled to said air block, wherein, when said trigger is actuated, said air block allows air to flow to said pneumatic cylinder through said air block, thereby actuating said pneumatic cylinder.

45. The device of claim 32, wherein said moveable hammer is pivotally coupled to said housing.

46. The device of claim 44, wherein said trigger is pivotally coupled to said housing.

47. The device of claim 32, further comprising a guide tube having an angled tip, at least a portion of said fracture pin being positioned within said guide tube.

48. The device of claim 32, wherein said fracture pin is removably coupled to said housing and said fracture pin is disposable.

49. The device of claim 47, wherein said angled tip may have an angle that ranges from approximately 30-60 degrees.

50. The device of claim 47, wherein said guide tube has an outside diameter that ranges from approximately 6-8 millimeters.

51. The device of claim 32, further comprising means for limiting the movement of said sharpened tip when said cylinder is actuated to thereby limit a depth of said at least one hole.

52. The device of claim 32, wherein said fracture pin is operatively coupled to said housing.

53. The device of claim 32, wherein said device further comprises a biasing member that is adapted to cause said sharpened tip to return to an initial, retracted position after said at least one hole has been formed.

54. A device for forming multiple holes in subchondral bone, comprising:
a housing;
a fracture pin having a sharpened tip, said sharpened tip adapted to penetrate subchondral bone; and
means for causing said sharpened tip to move and penetrate into said subchondral bone, thereby forming at least one of said holes.

55. The device of claim 54, wherein said means for causing said sharpened tip to move and penetrate into said subchondral bone comprises a cylinder that is adapted to, when actuated, cause said sharpened tip to move and penetrate into said subchondral bone, thereby forming at least one of said holes.

56. The device of claim 54, wherein said means for causing said sharpened tip to move and penetrate into said subchondral bone further comprises:
a hammer that is pivotally coupled to a rod of said cylinder;
an air block that is operatively coupled to said cylinder; and
a trigger that is operatively coupled to said air block, wherein, when said trigger is actuated, said air block allows air to flow to said cylinder through said air block to thereby actuate said cylinder.

57. The device of claim 54, wherein said means for causing said sharpened tip to move and penetrate into said subchondral bone comprises a movable hammer that is adapted to, when actuated, cause said sharpened tip to move and penetrate into said subchondral bone, thereby forming at least one of said holes.

58. The device of claim 54, wherein said means for causing said sharpened tip to move and penetrate into said subchondral bone further comprises a cylinder coupled to said moveable hammer.

59. The device of claim 54, wherein means for causing said sharpened tip to move and penetrate into said subchondral bone further comprises:
a trigger that is operatively coupled to said hammer, said hammer being rotatably moveable by actuation of said trigger; and
a hammer biasing spring being operatively coupled to said hammer.
